(19) United States
(12) Patent Application Publication
(10) Pub. No.: US 2024/0287279 A1
(43) Pub. Date: Aug. 29, 2024

(54) METHOD FOR PRODUCING A PURIFIED AND DECOLOURISED DIESTER MONOMER, BYMEANS OF DEPOLYMERISATION OF A POLYESTER FEEDSTOCK

(71) Applicants: IFP ENERGIES NOUVELLES, RUEIL-MALMAISON (FR); JEPLAN, INC., Kanagawa (FR)

(72) Inventors: DAVID CHICHE, RUEIL-MALMAISON CEDEX (FR); Guillaume BLANCKE, RUEIL-MALMAISON CEDEX (FR); Damien LEINEKUGEL LE COCQ, RUEIL-MALMAISON CEDEX (FR); Frederic FAVRE, RUEIL-MALMAISON CEDEX (FR); Yacine HAROUN, RUEIL-MALMAISON CEDEX (FR); Olivier THINON, RUEIL-MALMAISON CEDEX (FR); Adrien MEKKI-BERRADA, RUEIL-MALMAISON CEDEX (FR); Mayara AZIN GONDIM PAIVA, RUEIL-MALMAISON CEDEX (FR); Charles BONNIN, RUEIL-MALMAISON CEDEX (FR); Cyprien CHARRA, RUEIL-MALMAISON CEDEX (FR)

(73) Assignees: IFP ENERGIES NOUVELLES, RUEIL-MALMAISON (FR); JEPLAN, INC., Kanagawa (JP)

(21) Appl. No.: 18/571,049

(22) PCT Filed: Jun. 7, 2022
(86) PCT No.: PCT/EP2022/065429

§ 371 Date: Dec. 15, 2023

(30) Foreign Application Priority Data
Jun. 17, 2021 (FR) FR2106440

(51) Int. Cl. C08J 11/24; C07C 67/52

(52) U.S. Cl. CPC C08J 11/24; C07C 67/52; C07C 2601/16; C08J 2367/03

(57) ABSTRACT

The present invention relates to a process for depolymerization of a polyester feedstock comprising PET, comprising:
a) a step for conditioning the polyester feedstock, to produce a conditioned feedstock stream;
b) a step of depolymerization of the conditioned feedstock stream, at a temperature of between 150 and 300° C., in the presence of diol with a ratio by weight between the diol and the diester in the feedstock of between 0.3 and 8.0, to produce a reaction effluent;
c) a step of separation of the diol from the reaction effluent, to produce at least a liquid monomers effluent;
d) a step of separation of the liquid monomers effluent into a heavy impurities effluent and a prepurified monomers effluent; and
e) a step of purification of the prepurified monomers effluent, comprising a substep e1) of adsorption at a temperature of between 50 and 200° C. and a crystallization substep e2), and producing at least one decolourized purified diester monomer effluent.

### TECHNICAL FIELD

The invention relates to a process for preparing a diester monomer effluent by depolymerization by glycolysis of a polyester feedstock comprising in particular coloured and/or opaque and/or multilayer polyethylene terephthalate (PET), with a view to the recycling thereof into a polymerization unit. More particularly, the invention relates to a process for depolymerization by glycolysis of a polyester feedstock preferably comprising at least coloured and/or opaque PET, with a step of purification of the diester effluent comprising a step of adsorption followed by a step of crystallization of the diester monomer, to obtain a purified and decolourized diester monomer effluent.

### PRIOR ART

The chemical recycling of polyester, in particular of polyethylene terephthalate (PET), has been a subject of numerous studies aimed at breaking down the polyester, recovered in the form of waste, into monomers which will be able to be used again as feedstock for a polymerization process.

Numerous polyesters result from networks for collecting and sorting materials. In particular, polyester, in particular PET, may originate from the collection of bottles, container trays, films, resins and/or fibres composed of polyester (for instance textile fibres, tyre fibres). The polyester resulting from collecting and sorting channels is referred to as polyester to be recycled.

PET to be recycled can be classified into four main categories:
clear PET, predominantly composed of colourless transparent PET (generally at least 60% by weight) and azure coloured transparent PET, which does not contain pigments and can be used in mechanical recycling processes;
dark or coloured (green, red, etc.) PET, which can generally contain up to 0.1% by weight of dyes or pigments but remains transparent or translucent;
opaque PET, which contains a significant amount of pigments at contents typically varying between 0.25% and 5.0% by weight in order to opacity the polymer. Opaque PET is increasingly being used, for example in the manufacture of food containers, such as milk bottles, in the composition of cosmetic, plant-protection or dye bottles;
multilayer PET, which comprises layers of polymers other than PET or a layer of recycled PET between layers of virgin PET (that is to say, PET which has not undergone recycling), or a film of aluminium, for example. Multilayer PET is used, after thermoforming, to produce packagings, such as container trays.

The collection channels, which supply the recycling channels, are structured differently from one country to another. They are changing so as to maximize the amount of plastic upgraded from waste as a function of the nature and amount of the streams and of the sorting technologies. The channel for recycling these streams generally consists of a first step of conditioning in the form of flakes during which bales of raw packaging are washed, purified and sorted, ground and then purified again and sorted to produce a stream of flakes generally containing less than 1% by mass of “macroscopic” impurities (glass, metals, other plastics, wood, paper, cardboard, inorganic elements), preferentially less than 0.2% of “macroscopic” impurities and even more preferentially less than 0.05%.

Clear PET flakes may subsequently undergo an extrusion-filtration step to produce extrudates which can subsequently be reused as a mixture with virgin PET to produce new products (bottles, fibres, films). A step of solid state polymerization under vacuum (known by the abbreviation SSP) is necessary for food uses. This type of recycling is known as mechanical recycling.

Dark (or coloured) PET flakes can also be recycled mechanically. However, the colouration of the extrudates formed from the coloured streams limits the uses: dark PET is generally used to produce packaging straps or fibres. The outlets are thus more limited in comparison with those of clear PET.

The presence of opaque PET containing pigments at high contents, in PET to be recycled, presents problems to recyclers as opaque PET adversely affects the mechanical properties of recycled PET. Opaque PET is currently collected with coloured PET and is found in the coloured PET stream. In view of the development of the uses for opaque PET, the contents of opaque PET in the stream of coloured PET to be recycled are currently between 5-20% by weight and are tending to increase. In a few years' time, it will be possible to achieve contents of opaque PET in the coloured PET stream of greater than 20-30% by weight. However, it has been shown that, above 10-15% of opaque PET in the coloured PET streams, the mechanical properties of the recycled PET are adversely affected (cf. Impact du ddveloppement du PET opaque blanc sur le recyclage des emballages en PET [Impact of the growth of white opaque PET on the recycling of PET packagings], preliminary report of COTREP of 5/12/13) and prevent recycling in the form of fibres, the main outlet of the channel for coloured PET.

Dyes are natural or synthetic substances which are soluble, in particular in the polyester material, and are used to colour the material into which they are introduced. The dyes generally used have different natures and often contain heteroatoms of O and N type, and conjugated unsaturations, such as, for example, quinone, methine or azo functions, or molecules such as pyrazolone and quinophthalone. Pigments are finely divided substances which are insoluble, in particular in the polyester material, and which are used to colour and/or opacify the material into which they are introduced. The main pigments used to colour and/or opacify the polyesters, in particular PET, are metal oxides, such as TiO₂, CoAl₂O₄ or Fe₂O₃, silicates, polysulfides and carbon black. The pigments are particles with a size generally of between 0.1 and 10 μm and predominantly between 0.4 and 0.8 μm. The complete removal of these pigments, which is necessary in order to envisage recycling the opaque PET, by filtration is technically difficult as they have an extremely high clogging capability.

The recycling of coloured and opaque PETs is thus extremely problematic.

Patent application US 2006/0074136 describes a process for depolymerization by glycolysis of coloured PET, in particular resulting from the recovery of green-coloured PET bottles. The feedstock treated by this process takes the form of coloured PET flakes and is contacted with ethylene glycol in a reactor at a temperature of between 180 and 280° C. for several hours. The product of glycolysis which is obtained on conclusion of the depolymerization step is purified by passage over activated carbon at a temperature of more than 170° C. and then by extraction of the residual dyes, particularly yellow-coloured dyes, with a solvent, which may be an alcohol such as methanol or a glycol such as ethylene glycol. The BHET crystallizes in the extraction solvent and is then separated by filtration.

In patent application US 2015/0105532, the post-consumer PET, which comprises a mixture of different PETs, such as clear PET and coloured PETs such as blue PET, green PET and/or amber PET, in the form of flakes, is depolymerized by glycolysis in the presence of ethylene glycol and of an amine catalyst, in a reactor at 150-250° C., in batch mode. The resulting diester monomer is purified by direct filtration, then by adsorption on activated carbon and lastly by passage over ion-exchange resin, in particular at a temperature of 80-90° C., before being crystallized and recovered by filtration. Patent application US 2015/0105532 discloses another method for purification of the diester monomer obtained by short-path distillation at 200° C. Patent U.S. Pat. No. 6,642,350 in turn describes the purification of a crude BHET solution dissolved in methanol or ethylene glycol, comprising at least successively contacting said solution with an activated carbon, an anion-exchange resin and a cation-exchange resin, at a temperature of between 40 and 120° C., in particular of equal to 60° C., 65° C. or 80° C. Specifically, this patent shows that contacting with activated carbon alone under the conditions described above is not enough in particular to entirely decolourize the solution, since a residual colour, in particular yellow, persists, while the yellow colouring no longer appears after a succession of passages over activated carbon and anion- and cation-exchange resins.

In patent EP0865464, the process for depolymerization of polyester, in particular of coloured polyester, for example green PET, comprises the steps of depolymerization in the presence of a diol, in particular ethylene glycol, in a reactor at a temperature of between 180 and 240° C., optionally evaporation in a thin-film evaporator, dissolution in a hot solvent and a step of filtration to separate off insoluble impurities with a size of greater than 50 μm. The low proportion of pigments in coloured PET enables separation by filtration. However, this technology cannot operate with the amount of pigments present in opaque PET, since these pigments rapidly clog the filter.

Patent JP3715812 describes the production of refined BHET from PET in flake form. The depolymerization step consists of the glycolysis of the PET flakes, which have been pretreated beforehand by washing with water in solid form, in the presence of ethylene glycol and of a catalyst in a stirred reactor at 180° C. to remove the residual water, and then at 195-200° C. The depolymerization is followed by a step of prepurification by cooling, filtration, adsorption and treatment on ion-exchange resin, this step being presented as being very important and being carried out before the evaporation of the glycol and the purification of the BHET. According to JP3715812, the prepurification makes it possible to prevent the repolymerization of the BHET in the subsequent purification steps. However, passing through a step of filtration and ion-exchange resin may be extremely problematic when the feedstock comprises a large amount of very small solid particles, such as pigments and/or polymer compounds other than PET, such as for example polyolefins or polyamides, which is the case when the treated feedstock comprises opaque PET and/or multilayer preformed PET, in particular in substantial proportions (more than 10% by weight of opaque PET and/or of multilayer preformed PET). In parallel, patent EP 1 120 394 discloses a process for depolymerization of a polyester comprising a step of glycolysis in the presence of ethylene glycol and a process for purification of a solution of BHET on a cation-exchange resin and an anion-exchange resin.

Lastly, patent application FR 3053691 describes a process for depolymerization of a polyester feedstock comprising opaque PET and in particular from 0.1% to 10% by weight of pigments, by glycolysis in the presence of ethylene glycol. A purified bis(2-hydroxyethyl) terephthalate (BHET) effluent is obtained after particular steps of separation and of purification by adsorption. However, the BHET effluent obtained by the depolymerization process described in patent application FR 3053691 may have imperfections: the BHET effluent obtained undergoes in particular rapid colouration, despite being passed through a column of adsorbent.

The present invention aims to improve these processes for depolymerization by glycolysis of a polyester feedstock comprising PET, and preferably coloured and/or opaque PET, and in particular the process described in patent application FR 3053691, in order to improve the purification, and more particularly the decolourization, of the diester effluent obtained after separation of the heavy and solid impurities, such as the oligomers and the pigments. The object of the invention is specifically that of obtaining a diester effluent, in particular a BHET effluent, by depolymerization of a polyester feedstock comprising PET, and preferably coloured and/or opaque PET, with a high purity and a decolourized appearance.

### SUMMARY OF THE INVENTION

The aim of the invention is therefore a process for depolymerization of a polyester feedstock comprising polyethylene terephthalate, the process comprising:
a) a conditioning step fed at least with said polyester feedstock, to produce a conditioned feedstock stream;
b) a step of depolymerization by glycolysis, which is fed at least with the conditioned feedstock stream, and is operated at a temperature of between 150 and 300° C., with a residence time of between 0.1 and 10 h, in the presence of diol with a ratio by weight between the total amount of diol present in step b) and the amount of diester contained in the conditioned feedstock stream of between 0.3 and 8.0, to produce a reaction effluent;
c) a step of separation of the diol, which is fed at least with the reaction effluent obtained from step b), and is operated at a temperature of between 60 and 250° C. and at a pressure lower than that of step b), to produce at least a diol effluent and a liquid monomers effluent, said step of separation of the diol being implemented in a gas-liquid separation section or a succession of from two to five successive gas-liquid separation sections, each of the gas-liquid separation sections producing a gas effluent and a liquid effluent, the liquid effluent from the preceding section feeding the subsequent section, the liquid effluent obtained from the final gas-liquid separation section constituting the liquid monomers effluent, the gas effluent(s) being recovered so as to constitute said diol effluent(s);
d) a step of separation of the liquid monomers effluent obtained from step c) into a heavy impurities effluent and a prepurified monomers effluent, which is operated at a temperature of less than 250° C. and a pressure of less than 0.001 MPa, with a liquid residence time of less than 10 min; and
e) a step of purification of the prepurified monomers effluent, comprising an adsorption substep
e1) and a crystallization substep e2), and producing at least one decolourized purified diester monomer effluent, wherein
adsorption substep e1) is operated at a temperature of between 50 and 200° C. and a pressure of between 0.1 and 1.0 MPa and implements at least one section for mixing with a solvent and at least one section for adsorption in the presence of at least one adsorbent, crystallization substep e2) implements a solids production section, operated at a temperature of between 0 and 100° C. and at a pressure of between 0.00001 and 1.00 MPa, followed by a solid-liquid separation section.

Preferably, step e) of purification of the prepurified monomers effluent, of the process according to the invention, comprises an adsorption substep e1) followed by a crystallization substep e2), producing at least one decolourized purified diester monomer effluent and a spent solvent effluent, wherein

adsorption substep e1) is operated at a temperature of between 50 and 200° C. and a pressure of between 0.1 and 1.0 MPa and implements at least one section for mixing the prepurified monomers effluent obtained from step d) with a solvent and at least one section for adsorption in the presence of at least one adsorbent, to obtain an adsorption-pretreated monomer effluent, crystallization substep e2) implements a solids production section, fed at least with the adsorption-pretreated monomer effluent and operated at a temperature of between 0 and 100° C. and at a pressure of between 0.00001 and 1.00 MPa, followed by a solid-liquid separation section, to produce the decolourized purified diester monomer effluent and the spent solvent effluent.

One advantage of the present invention resides in obtaining, from a polyester feedstock comprising at least polyethylene terephthalate (PET), in particular coloured and/or opaque PET, a diester monomers effluent, in particular a bis(2-hydroxyethyl) terephthalate (BHET) effluent, which is purified and decolourized, and more particularly a white-coloured, purified solid diester monomers effluent, exhibiting colour parameters expressed in the CIE 1976 L*a*b* reference system, determined by colourimetry (in accordance with the method ASTM D6290 2019), preferably with:
a lightness (or luminance) parameter L* of close to 100, more particularly greater than 90.00 and preferably greater than 92.00 (100.00 being the maximum);
a parameter a* (corresponding to a green-red axis) of close to 0, more particularly between −1.50 and +1.50 and preferably between −1.00 and +1.00; and
a parameter b* (corresponding to a blue-yellow axis) of close to 0, more particularly between −2.50 and +2.50, more particularly between −1.00 and +1.50.

Advantageously, the process according to the invention makes it possible to obtain a diester monomers effluent, in particular a bis(2-hydroxyethyl) terephthalate (BHET) effluent, which is purified and decolourized, not exhibiting any significant absorption band (that is to say distinguishable from background noise) within the range of visible wavelengths, that is to say between 400 and 800 nm, when it is characterized by UV-visible spectrometry.

An advantage of the invention is therefore that it is able to process any type of polyester waste, waste which increasingly comprises pigments and dyes, such as coloured, opaque and even multilayer PETs. The process according to the invention, which is in particular suitable for processing opaque PET, makes it possible to remove the pigments and dyes and regain the diester monomer, in particular the bis(2-hydroxyethyl) terephthalate (BHET) monomer, by chemical reaction and particular purification steps. The diester monomer obtained may then be repolymerized to give a polymer which exhibits no difference from a virgin polyester, in particular a virgin PET, thus allowing access to all of the uses of virgin PET.

### LIST OF FIGURES

FIG. 1 represents a particular embodiment of the invention. A polyester feedstock 1 comprising coloured and/or opaque PET is conditioned in a conditioning step a). Step a) is also fed with a diol stream 11. The conditioned feedstock stream 2 is introduced into a step b) of depolymerization by glycolysis, which is likewise fed with another diol stream 12.

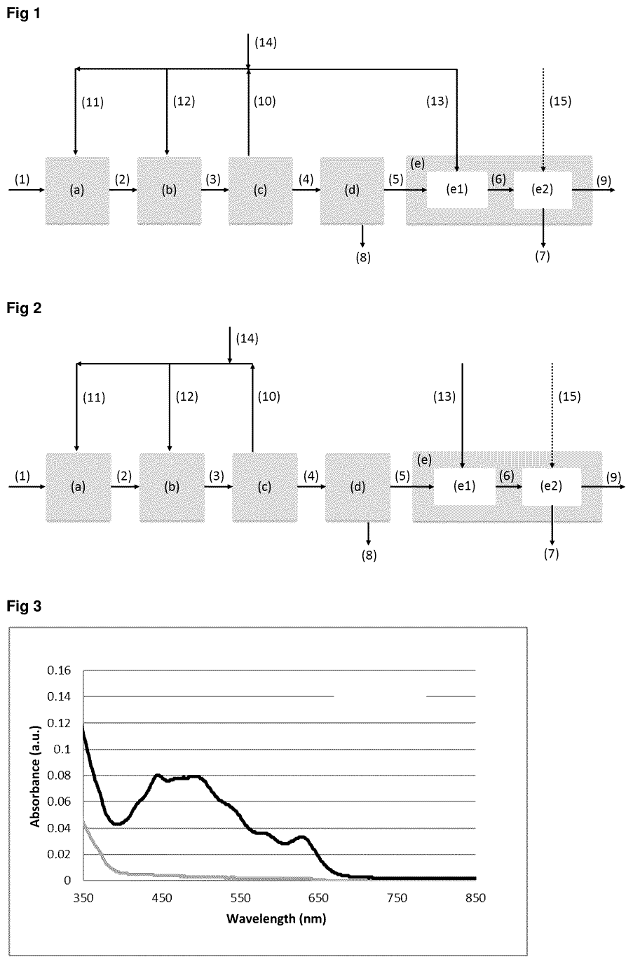

The reaction effluent 3 obtained after depolymerization is sent to a step c) of separation of the diol, which produces a liquid monomers effluent 4 and a diol effluent 10. The liquid monomers effluent 4 is sent to step d) of separation of the BHET. A supply of fresh diol 14, external to the process, is added to the diol effluent 10 recovered on conclusion of step c), before division into a diol stream 11 which feeds step a), another diol stream 12 which feeds step b) and a third diol stream 13 which feeds step e).

Step d) implements, in particular, a short-path evaporator for producing a prepurified monomers effluent 5 and a heavy impurities effluent 8. The heavy impurities effluent 8 may be at least partly recycled to the reaction step (step b). The prepurified monomers effluent 5 is sent to a purification step e).

In purification step e), said prepurified monomers effluent 5 feeds an adsorption section e1) which is also fed with a diol stream 13, to produce an adsorption-pretreated monomer effluent 6. The adsorption-pretreated monomer effluent 6 then feeds a crystallization section e2), in which the crystallization of the diester monomer and then the separation of the crystals formed are implemented, to produce a decolourized purified diester monomer effluent 7 and a spent diol solvent stream 9. Optionally, the crystallization section may also be fed with a crystallization solvent 15, for example with a stream of diol or water. The spent diol solvent stream 9 may optionally be recovered and recycled, in whole or in part, to steps e1), a) and/or b), and/or optionally e2), it being possible for the recovered spent diol solvent stream 9 to optionally be purified before recycling.

FIG. 2 represents a particular embodiment of the invention. A polyester feedstock 1 comprising coloured and/or opaque PET is conditioned in a conditioning step a). Step a) is also fed with a diol stream 11. The conditioned feedstock stream 2 is introduced into a step b) of depolymerization by glycolysis, which is likewise fed with another diol stream 12.

The reaction effluent 3 obtained after depolymerization is sent to a step c) of separation of the diol, which produces a liquid monomers effluent 4 and a diol effluent 10. The liquid monomers effluent 4 is sent to step d) of separation of the BHET. A supply of fresh diol 14, external to the process, is added to the diol effluent 10 recovered on conclusion of step c), before division into a diol stream 11 which feeds step a) and another diol stream 12 which feeds step b).

Step d) implements, in particular, a short-path evaporator for producing a prepurified monomers effluent 5 and a heavy impurities effluent 8. The heavy impurities effluent 8 may be at least partly recycled to reaction step b). The prepurified monomers effluent 5 is sent to a purification step e).

Purification step e) comprises:
an adsorption section e1) fed with said prepurified monomers effluent 5 and a solvent stream 13 external to the process, in particular a stream of water, to produce an adsorption-pretreated monomer effluent 6; and
a crystallization section e2) fed with the adsorption-pretreated monomer effluent 6, and optionally with a crystallization solvent, for example with a stream of water, and implementing the crystallization of the diester monomer and then the separation of the crystals formed, to produce a decolourized purified diester monomer effluent 7 and a spent solvent stream 9, in particular a water stream. Optionally, the crystallization section may also be fed with a crystallization solvent 15, for example with a stream of diol or water. The spent solvent stream 9 may optionally be recovered and recycled, in whole or in part, to the section e1), and/or optionally to the section e2), it being possible for the recovered spent solvent stream to optionally be purified before recycling.

FIG. 3 shows the UV-visible spectra determined for the solids obtained by the processes described in example 1 (in grey) and in example 2 (in black).

### DESCRIPTION OF THE EMBODIMENTS

According to the invention, polyethylene terephthalate or poly(ethylene terephthalate), also simply called PET, has an elementary repeating unit which comprises a diester (in particular a terephthalic acid diester) and is of formula:

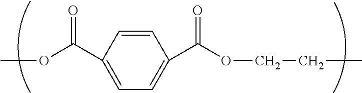

Conventionally, PET is obtained by polycondensation of terephthalic acid (PTA) or dimethyl terephthalate (DMT) with ethylene glycol.

In the continuation of the text, the expression “per mole of diester in said polyester feedstock” corresponds to the number of moles of —[O—CO—O—(C₆H₄)—CO—O—CH₂—CH₂]— unit in said polyester feedstock, in particular in the PET included in the polyester feedstock, which is the diester unit obtained notably from the reaction of PTA and ethylene glycol, in the PET included in said polyester feedstock.

According to the invention, the term “monomer” or “diester monomer” advantageously denotes the repeating unit of a polyester in the polyester feedstock, in particular of the polyethylene terephthalate PET in the polyester feedstock, and defines a diester of a dicarboxylic acid, preferably of an aromatic dicarboxylic acid and preferentially of terephthalic acid, and of a diol comprising preferably between 2 and 12 carbon atoms, preferentially between 2 and 4 carbon atoms, the preferred diol being ethylene glycol. More particularly, the “monomer” or “diester monomer” corresponds to the product targeted by the process according to the invention. Thus, according to one embodiment of the invention, the “monomer” or “diester monomer”, the product targeted by the invention, has a chemical formula of the type: HOCₙH₂ₙ—CO₂—(Aro)—CO₂—CₙH₂ₙOH, with n=2-12, preferably n=2-4 and —(Aro)—═—(C₆H₄)— representing an aromatic ring. Preferably, the term “monomer” or “diester monomer” denotes bis(2-hydroxyethyl) terephthalate (BHET), which is the target product of the depolymerization of PET in the presence of ethylene glycol, of chemical formula HOC₂H₄—CO₂—(C₆H₄)—CO₂—C₂H₄OH, in which —(C₆H₄)— represents an aromatic ring.

The term “oligomer” typically denotes a polymer of small size, consisting generally of 2 to 20 elementary repeating units. According to the invention, the term “ester oligomer” or “BHET oligomer” denotes a terephthalate ester oligomer comprising between 2 and 20, preferably between 2 and 5, elementary repeating units of formula —[O—CO—(C₆H₄)—CO—O—C₂H₄]—, where —(C₆H₄)— is an aromatic ring.

According to the invention, the terms “diol” and “glycol” are used without distinction and correspond to compounds comprising 2 hydroxyl —OH groups and preferably comprising between 2 and 12 carbon atoms, preferentially between 2 and 4 carbon atoms. The preferred diol is ethylene glycol, also referred to as monoethylene glycol or MEG.

Therefore, the diol or diol effluent streams used in the steps of the process of the invention preferably comprise ethylene glycol (or MEG), advantageously in an amount of greater than 40% by weight, preferentially greater than 50% by weight, preferably greater than or equal to 60% by weight, of the total weight of said diol or diol effluent stream.

The term “dye” defines a substance that is soluble in the polyester material and that is used to colour it. The dye can be of natural or synthetic origin.

According to the invention, the term “pigment”, more particularly opacifying and/or colouring pigment, defines a finely divided substance which is insoluble in particular in the polyester material. The pigments are in the form of solid particles with a size of generally between 0.1 and 10 μm, and predominantly between 0.4 and 0.8 μm. They are often of inorganic nature. The pigments generally used, in particular for opacifying, are metal oxides, such as TiO₂, CoAl₂O₄ or Fe₂O₃, silicates, polysulfides and carbon black.

According to the present invention, the expressions “of between . . . and . . . ” and “between . . . and . . . ” are equivalent and mean that the limit values of the interval are included in the described range of values. If such were not the case and if the limit values were not included in the described range, such a clarification will be given by the present invention.

For the purposes of the present invention, the various ranges of parameters for a given step, such as the pressure ranges and the temperature ranges, may be used alone or in combination. For example, within the meaning of the present invention, a range of preferred pressure values can be combined with a range of more preferred temperature values.

In the text hereinbelow, particular embodiments of the invention may be described. They may be implemented separately or combined together without limitation of combinations when this is technically feasible.

The terms “upstream” and “downstream” should be understood as a function of the general flow of the stream in the process.

According to the present invention, the pressures are absolute pressures and are given in MPa or MPa absolute (or MPa abs).

### Feedstock

The process according to the invention is fed with a polyester feedstock comprising polyethylene terephthalate (PET), preferably comprising at least opaque PET, coloured PET, multilayer PET or mixtures thereof, with preference at least opaque PET and/or coloured PET, optionally multilayer PET.

Said polyester feedstock is advantageously a polyester feedstock to be recycled, obtained from waste, in particular plastic waste, collection and sorting channels. Said polyester feedstock may originate, for example, from the collection of bottles, container trays, films, resins and/or fibres consisting of polyethylene terephthalate.

The polyester feedstock advantageously comprises at least 50% by weight, preferably at least 70% by weight, and with preference at least 90% by weight of polyethylene terephthalate (PET), the maximum being 100% by weight of PET.

Said polyester feedstock preferably comprises at least one PET selected from opaque, dark or coloured and multilayer PET and mixtures thereof. Very particularly, said polyester feedstock comprises at least 10% by weight of opaque PET, very preferably at least 15% by weight of opaque PET, said opaque PET advantageously being opaque PET to be recycled, i.e. PET obtained from collection and sorting channels. The polyester feedstock may comprise 100% by weight of opaque PET. More particularly, it may comprise up to 70% by weight of opaque PET.

Said polyester feedstock advantageously comprises between 0.1% and 10% by weight of pigments, advantageously between 0.1% and 5% by weight. It also preferably comprises between 0.005% and 1% of dyes, in particular between 0.01% and 0.20% by weight.

In collection and sorting channels, polyester waste is washed and ground before constituting the polyester feedstock of the process according to the invention.

The polyester feedstock may be totally or partly in the form of flakes, the greatest length of which is less than 10 cm, preferentially between 5 and 25 mm, or in micronized solid form, i.e. in the form of particles preferably between 10 micrometres (μm) and 1 mm in size. The feedstock may also comprise “macroscopic” impurities, preferably less than 5% by weight, preferentially less than 3% by weight of “macroscopic” impurities, such as glass, metal, plastics other than polyester (for example PP, PEHD, etc.), wood, paper, cardboard or inorganic elements. Said polyester feedstock may also be totally or partly in the form of fibres, such as textile fibres, which have optionally been pretreated to remove cotton or polyamide fibres, or any textile fibre other than polyester, or such as tyre fibres, which have optionally been pretreated notably to remove polyamide fibres or rubber or polybutadiene residues. Said polyester feedstock may also comprise polyester obtained from production rejects of polyester polymerization and/or transformation processes. The polyester feedstock may also comprise elements used as polymerization catalyst and as stabilizers in PET production processes, such as antimony, titanium or tin.

### Conditioning Step a)

Said process according to the invention comprises a conditioning step a), fed at least with said polyester feedstock, and producing a conditioned feedstock stream.

Said step a) makes it possible in particular to heat and pressurize said polyester feedstock to the operating conditions of the depolymerization step b).

In the conditioning step a), the polyester feedstock is progressively heated to a temperature close to or even slightly above its melting point, so as to become at least partly liquid. Advantageously, at least 70% by weight of the polyester feedstock, very advantageously at least 80% by weight, preferably at least 90% by weight, preferentially at least 95% by weight of the polyester feedstock is in liquid form on conclusion of step a). The temperature at which step a) is implemented is advantageously between 200 and 300° C., preferably between 250 and 290° C. This temperature is kept as low as possible to minimize the thermal degradation of the polyester, but must be sufficient to at least partly melt the polyester feedstock.

The conditioning step a) may advantageously be operated under inert atmosphere, to limit the introduction of oxygen into the system and the oxidation of the polyester feedstock. Advantageously, step a) is implemented at a pressure preferably of between atmospheric pressure (i.e. 0.1 MPa) and 20 MPa, preferably between 0.15 MPa and 10 MPa.

Advantageously, step a) may also be fed with a diol stream, preferably an ethylene glycol stream, with a ratio by weight of the diol stream in relation to the polyester feedstock, that is to say a ratio between the flow rate by weight of the diol stream which feeds step a) and the flow rate by weight of the polyester feedstock which feeds step a), of between 0.03 and 6.00, preferably between 0.05 and 5.00, preferentially between 0.10 and 4.00, with preference between 0.50 and 3.00. Very advantageously, the diol stream which feeds step a) corresponds to at least a fraction of the, preferably purified, diol effluent obtained from step c), optionally as a mixture with a supply of fresh diol external to the process according to the invention. The effect of contacting the polyester feedstock with a diol stream is to initiate the depolymerization reaction of the polyester feedstock, before introduction into the depolymerization step b). It also makes it possible to reduce the viscosity of the polyester feedstock, which facilitates the homogenization of the feedstock-diol mixture and consequently the depolymerization reaction.

According to a preferred embodiment of the invention, step a) implements an extruder, optionally followed by at least one static or dynamic mixer.

Preferably, the residence time in said extruder, defined as the volume of said extruder divided by the volume flow rate of polyester feedstock, is advantageously less than or equal to 5 min, preferably less than or equal to 2 min, and with preference greater than 1 second, preferentially greater than or equal to 10 seconds. Advantageously, the extruder makes it possible to bring the polyester feedstock to a temperature of between 200 and 300° C., preferentially between 250 and 290° C., and to a pressure of preferably between atmospheric pressure (i.e. 0.1 MPa) and 20 MPa, preferably between 0.15 MPa and 10 MPa, conditions under which said polyester feedstock is advantageously at least partly molten.

The extruder is advantageously connected to a vacuum extraction system so as to remove impurities, such as dissolved gases, light organic compounds and/or moisture present in the feedstock. A filtration system may also advantageously be implemented at the extruder outlet, and advantageously upstream of step b), to remove solid particles having a size of greater than 40 μm, preferably having a size of less than 2 cm, such as sand particles. The feeding of the polyester feedstock into the extruder is carried out advantageously by any methods known to a person skilled in the art, for example via a feed hopper, and may advantageously be inertized in order to limit the introduction of oxygen into the system.

The polyester feedstock may also advantageously be mixed, in the conditioning step a), with at least a fraction of the heavy impurities effluent obtained from step d).

The conditioned feedstock stream obtained from the conditioning section is advantageously sent to the depolymerization step b).

### Depolymerization Step b)

The process according to the invention comprises a step b) of depolymerization by glycolysis advantageously of the polyethylene terephthalate (PET) of the feedstock, in the presence of diol.

The diol present in step b) advantageously serves as depolymerization agent but also as solvent, thus making it possible to reduce the viscosity of the reaction medium and facilitating the reactions and hence the depolymerization. The diol present in step b) is introduced in step a) or in step b), or else in step a) and in step b). The diol is advantageously monoethylene glycol.

Depolymerization step b) is fed at least with the conditioned feedstock stream obtained from conditioning step a) and optionally with a supply of diol obtained in particular from a diol effluent internal or external to the process according to the invention, such that the ratio by weight between the total amount by weight of diol present in step b), corresponding to the sum of the amounts by weight of diol introduced in step a) and/or in step b), and the amount by weight of diester contained in the conditioned feedstock stream (i.e. contained in the polyester feedstock, in particular in the PET of the polyester feedstock, the amount by weight of diester thus corresponding more precisely to the weight of polyester, in particular PET, in the polyester feedstock), is between 0.3 and 8.0, preferably between 1.0 and 7.0, with preference between 1.5 and 6.0. In other words, depolymerization step b) is fed with the conditioned feedstock stream obtained from conditioning step a) and optionally with a supply of diol, such that the molar ratio between the total molar amount of diol introduced in step a) and/or in step b) in relation to the total molar amount of diester contained in the conditioned feedstock stream (i.e. contained in the polyester feedstock) is respectively between 1.0 and 24.0, preferably between 3.0 and 21.0, with preference between 4.5 and 18.0.

Preferably, depolymerization step b) is fed with the conditioned feedstock stream obtained from step a) and with a supply of diol, preferably a supply of ethylene glycol, advantageously obtained from a diol effluent internal or external to the process according to the invention, such that the ratio by weight between the total amount of diol introduced in step b) and optionally in step a) in relation to the total amount of diester contained in the conditioned feedstock stream (i.e. contained in the polyester feedstock and hence more precisely the weight of polyester, in particular of PET, in the polyester feedstock) is between 0.3 and 8.0, preferably between 1.0 and 7.0, with preference between 1.5 and 6.0 (i.e. a molar ratio of diol in relation to the diester respectively of between 1.0 and 24.0, preferably approximately between 3.0 and 21.0, with preference between 4.5 and 18.0).

Advantageously, depolymerization step b) implements one or more reaction sections, preferably at least two reaction sections, with preference between two and four reaction sections, preferably functioning in series. Each reaction section may comprise a reactor, more particularly any type of reactor known to a person skilled in the art which makes it possible to perform a depolymerization or transesterification reaction, and preferably a reactor stirred with a mechanical stirring system and/or with a recirculation loop and/or by fluidization. In each reaction section, the reactor may optionally comprise a conical base which makes it possible to bleed off the impurities. With preference, said depolymerization step b) implements at least two reaction sections, preferably between two and four reaction sections, functioning in series, the reaction section(s), starting from the second reaction section, being operated at a mutually identical or different temperature which is lower than or equal to the temperature of the first reaction section, preferably lower, and preferentially 10 to 50° C. lower, or even 20 to 40° C. lower, relative to the temperature of the first reaction section.

Depolymerization step b) is operated at a temperature of between 150 and 300° C., preferably between 180 and 290° C., with preference between 210 and 270° C., in particular in the liquid phase. Advantageously, step b) is implemented with a residence time in each reaction section of between 0.1 and 10 h, preferably between 0.25 and 8 h, between 0.5 and 6 h. The residence time in a reaction section is defined as the ratio of the liquid volume of said reaction section to the volume flow rate of the stream exiting said reaction section.

The operating pressure of the reaction section(s) of step b) is determined so as to keep the reaction system in the liquid phase. This pressure is advantageously at least 0.1 MPa, preferentially at least 0.4 MPa, and preferably less than 5 MPa. The term “reaction system” means all of the constituents and phases present in said step b).

The glycolysis reaction may be carried out in the presence or absence of a catalyst.

When the glycolysis reaction is carried out in the presence of a catalyst, the latter can be homogeneous or heterogeneous and chosen from the esterification catalysts known to a person skilled in the art, such as complexes, oxides and salts of antimony, tin or titanium, alkoxides of metals from groups (I) and (IV) of the periodic table of the elements, organic peroxides or acidic/basic metal oxides.

A preferred heterogeneous catalyst advantageously comprises at least 50% by mass relative to the total mass of the catalyst, preferentially at least 70% by mass, advantageously at least 80% by mass, very advantageously at least 90% by mass and even more advantageously at least 95% by mass of a solid solution consisting preferably of at least one spinel of formula ZₓAl₂O₍₃₊ₓ₎ in which x is between 0 (limit excluded) and 1, and Z is chosen from Co, Fe, Mg, Mn, Ti and Zn, and comprising no more than 50% by mass of alumina and of oxide of the element Z. Said preferred heterogeneous catalyst advantageously contains no more than 10% by mass of dopants chosen from silicon, phosphorus and boron, taken alone or as a mixture.

For example, and in a non-limiting manner, said solid solution may consist of a mixture of spinel ZnAl₂O₄ and of spinel CoAl₂O₄, or else may consist of a mixture of spinel ZnAl₂O₄, of spinel MgAl₂O₄ and of spinel FeAl₂O₄, or else may consist solely of spinel ZnAl₂O₄.

According to a particular embodiment of the invention, a catalyst, preferably chosen from amines, preferably tertiary mono- and diamines, such as for example tetramethylethylenediamine (TMEDA), pentamethyldiethylenetriamine (PMDETA), trimethyltriazacyclononane (TACN), triethylamine (TEA), 4-(N,N-dimethylamino)pyridine (DMAP), 1,4-diazabicyclo[2.2.2]octane (DABCO), N-methylimidazole (NMI), and alkali metal or alkaline earth metal hydroxides, such as for example Mg(OH)₂ and NaOH, may be added to the conditioned feedstock stream in depolymerization step b).

Said depolymerization step is preferably carried out without addition of external catalyst to the polyester feedstock.

Said depolymerization step may advantageously be carried out in the presence of a solid adsorbing agent in powder or shaped form, the function of which is to capture at least a portion of the coloured impurities, thus relieving the strain on the purification step e). Said solid adsorbing agent is advantageously an activated carbon.

Step b) may also be fed with at least a fraction of the heavy impurities effluent obtained from step d).

The glycolysis reaction makes it possible to convert the polyester of the polyester feedstock, in particular the PET of the polyester feedstock, and possibly its oligomers, into diester monomer and oligomers, and advantageously PET into at least the monomer bis(2-hydroxyethyl) terephthalate (BHET) and oligomers of BHET. The conversion of the polyester feedstock, more particularly of the PET of the polyester feedstock, in depolymerization step b) is greater than 50%, preferably greater than 70%, with preference greater than 85%. Advantageously, the molar BHET yield is greater than 50%, preferably greater than 70%, with preference greater than 85%. The molar BHET yield corresponds to the molar flow rate of BHET at the outlet of step b) to the number of moles of diester present in the polyester feedstock feeding said step a).

An internal recirculation loop may advantageously be implemented in step b), with withdrawal of a fraction of the reaction system from a reaction section, the filtration of this fraction and the reinjection of said filtered fraction into said reaction section of step b). This internal loop makes it possible to remove the “macroscopic” solid impurities that may be present in the reaction liquid.

Advantageously, the depolymerization step b) makes it possible to obtain a reaction effluent, advantageously in essentially liquid form, which is sent to a step c) of separation of the diol.

### Step c) of Separation of the Diol

The process according to the invention comprises a step c) of separation of the diol from the reaction effluent obtained from step b). Step c) is thus advantageously fed at least with the reaction effluent obtained from step b), and is operated at a temperature of between 60 and 250° C. at a pressure lower than that of step b), producing at least a diol effluent and a liquid monomers effluent. The main function of step c) is to recover all or part of the unreacted diol and/or diol generated during the depolymerization step.

Advantageously, step c) is operated at a pressure lower than that of step b), so as to vaporize a fraction of the reaction effluent from step b) to give one or more gas effluents. Said gas effluent(s) obtained on conclusion of step c) consist(s) of more than 40% by weight of diol, preferably more than 50% by weight of diol, with preference more than 60% by weight of diol, the preferred diol being ethylene glycol (MEG), and constitute(s) one or more diol effluents.

Advantageously, step c) implements a gas-liquid separation section or a succession of gas-liquid separation sections, advantageously from two to five successive separation sections, for example three gas-liquid separation sections. Each of the gas-liquid separation sections produces a liquid effluent and a gas effluent. The liquid effluent from the preceding section feeds the subsequent section. The liquid effluent obtained from the last gas-liquid separation section constitutes the liquid monomers effluent. The gas effluent(s) is/are recovered so as to constitute said diol effluent(s).

Advantageously, at least a fraction of at least one gas effluent produced may be condensed, in particular to give at least one liquid diol effluent. The diol effluent(s) may contain other compounds, such as dyes, light alcohols, water or diethylene glycol. All or part of the diol effluent(s) obtained from step c), kept in the gaseous state or condensed in liquid form, may be sent, each independently or as a mixture, to a step of purification of the diol to produce at least one purified diol effluent, prior to recycling thereof. Optionally, all or part of the diol effluent(s) obtained from step c), preferably after condensation, after purification or directly, may advantageously be recycled to step a) and/or step b) and/or sent to step e), optionally as a mixture with a supplement of diol external to the process according to the invention.

According to a preferred embodiment of the invention, the diol effluent(s), advantageously kept in the gaseous state and/or after condensation, obtained from step c) is/are sent to a purification step to produce, at least, a purified diol effluent prior to recycling thereof in whole or in part to steps a) and/or b), and/or to step e). In this embodiment, said step of purification of the diol effluent(s) may comprise, non-exhaustively, adsorption on a solid (for example on activated carbon) in order to remove the dyes, and/or one or more distillations in order to separate the impurities such as diethylene glycol, water and other alcohols.

Advantageously, at least one of the gas-liquid separation sections may be implemented in a falling-film evaporator or a thin-film evaporator. Step c) may also implements at least one separation section which carries out a short-path distillation.

Step c) is operated such that the temperature of the liquid effluents is kept above a low value below which the target diester monomer precipitates, and below a high value, which depends on the molar diol/monomer ratio, above which the diester monomer undergoes significant repolymerization. The operating temperature in step c) is between 60 and 250° C., preferably between 90 and 220° C., with preference between 100 and 210° C. Implementing a succession of gas-liquid separations, advantageously a succession of from 2 to 5 successive separations, is particularly advantageous since it makes it possible to adjust in each separation the temperature of the liquid effluent in correspondence with the abovementioned constraints.

The pressure in step c), preferably in each separation section, is advantageously adjusted to allow the evaporation of the diol at the temperature defined in each separation section, while minimizing the repolymerization and enabling optimum integration in terms of energy. It is generally between 0.00001 and 0.2 MPa, preferably between 0.00004 and 0.15 MPa, with preference between 0.00004 and 0.1 MPa.

The gas-liquid separation section(s) is/are advantageously stirred by any method known to a person skilled in the art.

### Step d) of Separation of the Monomer

The process according to the invention comprises a step d) of separation of the liquid monomers effluent obtained from step c) into a heavy impurities effluent and a prepurified monomers effluent.

Said step d) is advantageously operated at a temperature of less than 250° C., with preference less than 230° C., and very preferably less than 200° C., and preferably greater than 110° C., and a pressure of less than 0.001 MPa, preferably less than 0.0005 MPa, with preference less than 0.00005 MPa, and preferably greater than 0.000001 MPa, with a liquid residence time of less than 10 min, preferably less than 5 min, with preference less than 1 min, and preferably greater than 0.1 seconds.

The objective of this separation step d) is to separate the diester monomer, in particular the BHET, which is vaporized, from the oligomers not completely converted during the depolymerization step, which remain liquid and therefore also capture the heavy impurities, such as the pigments, and from the unconverted polyester polymer, from other polymers possibly present in the polyester feedstock, and from the polymerization catalysts, while minimizing the loss of monomers by repolymerization. Some oligomers may possibly be entrained with the monomer, in particular those having a small size (i.e. of low molar masses, such as for example dimers). The heavy impurities, such as for example the pigments, the unconverted polyester polymer, the other polymers possibly present in the polyester feedstock and the polymerization catalysts, are advantageously located with the oligomers in the heavy impurities effluent.

Owing to the possible presence in the polyester feedstock of polymerization catalysts, the separation must be carried out with very short liquid residence times and at a temperature of not more than 250° C., so as to limit any risk of repolymerization of the monomer, more particularly of BHET, during this step. A separation by simple atmospheric distillation cannot, therefore, be contemplated.

Advantageously, separation step d) implements a falling-film or thin-film evaporation system or a falling-film or thin-film short-path distillation system, preferably a falling-film or thin-film short-path distillation system.

A very low operating pressure, advantageously of less than 0.001 MPa, preferably of less than 0.0005 MPa, with preference less than 0.00005 MPa, and preferably of greater than 0.000001 MPa, is necessary in order to allow step d) to be operated at a temperature of less than 250° C., preferably less than 230° C., while allowing the monomer to vaporize.

A polymerization inhibitor may advantageously be mixed with the liquid monomers effluent before feeding said step d).

A flux may also advantageously be mixed with the liquid monomers effluent before feeding said step d), so as to facilitate the removal of the heavy impurities, for example the pigments, at the bottom of the short-path distillation or evaporation system. This flux must have a much higher boiling point than the target diester monomer, in particular than the BHET, under the operating conditions of step d). It may, for example, be polyethylene glycol, or PET oligomers.

In particular, the heavy impurities effluent comprises pigments, oligomers and possibly BHET which has not been separated out. The heavy impurities effluent is advantageously recycled, in whole or in part, to the conditioning step a) and/or to step b). A portion of said heavy impurities effluent may advantageously be recycled directly to step a) and/or step b), alone or as a mixture with a diol effluent. The heavy impurities effluent may advantageously undergo at least one purification step, preferably a filtration step, prior to the recycling thereof, so as to reduce the amount of pigments and/or other solid impurities. The portion of said separated heavy impurities effluent, having a high pigment content, may advantageously be purged from the process and sent to an incineration system. A fraction of said heavy impurities effluent is preferably recycled to step a) and/or step b) without prior separation of the solid impurities.

Said prepurified monomers effluent, also referred to as prepurified diester monomers effluent, obtained from the separation section of step d), is advantageously sent to purification step e).

Optionally, said prepurified monomers effluent obtained from the separation section of step d) may be sent, prior to step e), to a gas/liquid separation section, operated in any equipment known to a person skilled in the art, at a temperature of between 100 and 250° C., preferably between 110 and 200° C., and with preference between 120 and 180° C., and at a pressure of between 0.00001 and 0.1 MPa, preferably between 0.00001 and 0.01 MPa, and with preference between 0.00001 and 0.001 MPa. In a preferred embodiment of the invention, in which the separation step d) is implemented in a system of evaporation by falling-film or thin-film short-path distillation, said optional gas-liquid separation section is integrated into the evaporation system. Said optional gas-liquid separation section makes it possible to separate a gaseous diol effluent and a liquid prepurified monomer effluent, making it possible to further reduce the amount of diol remaining in the prepurified monomers effluent, or even to eliminate the residual diol, by recovering, in said gaseous diol effluent, more than 50% by weight, preferably more than 70% by weight, with preference more than 90% by weight of the diol possibly entrained in step d) with the prepurified monomers effluent. The amount of monomers entrained in said gaseous diol effluent is preferably less than 1% by weight, with preference less than 0.1% by weight and more preferably less than 0.01% by weight, relative to the amount by weight of monomers present in the prepurified monomers effluent. Said gaseous diol effluent is then advantageously condensed, optionally pretreated in a purification step alone or as a mixture with the diol effluent(s) obtained from step c), and recycled to step a) and/or to step b) and/or as a mixture into step e). In the case where the process comprises this optional gas-liquid separation section, what is sent to step e) is the liquid prepurified monomers effluent obtained at the end of said optional gas-liquid section.

### Purification Step e)

The process according to the invention comprises a step of purification of the prepurified monomers effluent obtained from step d), producing at least one decolourized purified diester monomer effluent and a spent solvent effluent.

Said purification step e) advantageously makes it possible to remove the residual dyes from the prepurified monomers effluent, in particular the dyes whose boiling point is less than the cut point, namely under the temperature and pressure conditions carried out in particular in step d) of separation of the monomer. The reason is that these residual dyes, entrained with the prepurified monomers effluent which they colour, can be removed effectively in said purification step e). Purification step e) also makes it possible to advantageously remove organic or inorganic, in particular colourless, residual impurities that may possibly still be present in the prepurified monomers effluent obtained from step d), such as residual salts, compounds derived from the diol dimer, in particular from the ethylene glycol dimer, i.e. compounds derived from diethylene glycol such as diethylene glycol esters (for example 2-(2-hydroxyethoxy)ethyl 2-hydroxyethyl terephthalate), and other comonomers of the diester monomer that have not been removed by distillation (for example positional isomers of BHET). Purification step e) comprises an adsorption substep e1) and a crystallization substep e2), the substeps e1) and e2) being described hereinafter. Preferably, purification step e) comprises an adsorption substep e1) followed by a crystallization substep e2).

### Adsorption Substep e1)

Advantageously, adsorption substep e1) implements at least one section for mixing the prepurified monomers effluent obtained from step d), or optionally the liquid prepurified monomer effluent, with a solvent and at least one adsorption section. Adsorption substep e1) makes it possible to obtain an adsorption-pretreated monomer effluent, which is advantageously at least partially decolourized.

The mixing section of substep e1) makes it possible to obtain a monomer-solvent mixture. It is fed with the prepurified liquid monomers effluent obtained from step d), or optionally the liquid prepurified monomer effluent, and a solvent, preferably chosen from water, alcohols, diols, and mixtures thereof, preferably from water, diols, for example ethylene glycol, and mixtures thereof. Preferably, the solvent which feeds the mixing section of substep e1) comprises, preferably consists of, water and/or a diol, more particularly the same diol as that used for the depolymerization by glycolysis, that is to say the same diol as that which feeds step a) and/or b), for example ethylene glycol.

Advantageously, the solvent which feeds the mixing section of substep e1) comprises, preferably consists of, a fraction of the diol effluent obtained from step c), all or part of an, optionally purified, solvent effluent obtained from the spent solvent effluent obtained at the outlet of the solid-liquid separation section of substep e2), a supply of solvent, preferably diol and/or water, external to the process according to the invention, or mixtures thereof. According to a particular embodiment of the invention, the solvent comprises, preferably consists of, all or part of a solvent effluent, which may or may not be purified, obtained from the spent solvent effluent obtained at the outlet of the solid-liquid separation section of substep e2), optionally supplemented by a supply of solvent external to the process according to the invention.

Preferably, the amount of solvent introduced into the mixing section of substep e1) is adjusted such that the prepurified monomers effluent, or optionally the liquid prepurified monomer effluent, represents between 20% and 90% by weight, preferentially between 30% and 80% by weight, preferably between 40% and 75% by weight and more preferably still between 40% and 60% by weight, of the total weight of the monomer-solvent mixture of said mixing section. Advantageously, the mixing section of substep e1) is operated at a temperature of between 50 and 200° C., preferably between 70 and 170° C., and with preference between 80 and 150° C., and at a pressure of between 0.1 and 1.0 MPa, preferably between 0.1 and 0.8 MPa, and with preference between 0.1 and 0.5 MPa. The solvent may be heated, prior to said mixing section, preferably to the temperature at which the mixing section is operated, in particular to a temperature of between 50 and 200° C., preferably between 70 and 170° C., and with preference between 80 and 150° C.

The mixing section of substep e1) may optionally implement a static or dynamic mixer, in particular a static mixer.

Advantageously, the monomer-solvent mixture obtained at the end of the mixing section of substep e1) feeds at least one adsorption section, preferably between one and ten, with preference between one and four adsorption sections. Each adsorption section of substep e1) is operated in the presence of at least one adsorbent, advantageously at a temperature of between 50 and 200° C., preferably between 70 and 170° C., preferentially between 80 and 150° C., and with preference between 80 and 120° C., and very advantageously at a pressure of between 0.1 and 1.0 MPa, in particular between 0.1 and 0.8 MPa, and more particularly between 0.1 and 0.5 MPa.

Each adsorption section advantageously comprises at least one adsorber (for example reactor or column), and preferably up to four adsorbers. Very advantageously, the residence time in each adsorber is between 20 minutes and 40 hours, preferably between 1 hour and 30 hours, preferably between 1 hour and 20 hours. The residence time is defined here as the ratio between the internal volume of the adsorber and the volume flow rate of the monomer-solvent mixture obtained from the mixing section. When the adsorption section(s) comprise(s) two or more adsorbers, that is to say between two and four adsorbers, the adsorbers are placed in series or in parallel with respect to one another, in each section.

Each adsorption section is operated in the presence of at least one adsorbent and preferably up to five different adsorbents. According to a very particular embodiment, each adsorption section implements one or two different adsorbents. According to the invention, adsorbents are said to be different when their nature and/or their composition and/or their different particle size and/or their textural characteristics, such as the pore volume, is/are different. Preferably, different adsorbents are of different nature. The reason is that it may be advantageous to combine two or more different adsorbents, in particular of different nature, in order to optimize the removal of the residual dyes, which may themselves be of very different nature. Indeed, since the polyester feedstock of the process is obtained from polyester waste, such as PET packaging or plastic bottle waste, it may comprise a very large number of coloured and/or opaque PETs and therefore a very large number of different dye compounds. The colouration of the effluent obtained from step d) may also originate from a degradation or transformation of compounds constituting the feedstock during the steps of conditioning a), depolymerization b), separation of the diol c) and separation of the monomer d).

When the adsorption section comprises between two and five different adsorbents, said different adsorbents are in a mixture or placed in series in said adsorption section, preferably in series and more preferentially each of the adsorbents is in different adsorbers (for example reactors or columns) placed in series or in parallel, preferably in series.

Advantageously, the adsorbent(s) is/are in particular in solid form. Preferably, the adsorbent(s) is/are chosen from activated carbons, alumina and clays. Activated carbons are for example obtained from petcoke, from bituminous coal or from any other fossil origin, or obtained from biomass such as wood, coconut or any other source of biomass. Different starting materials may also be mixed in order to obtain activated carbons which may be used as adsorbents in said adsorption section. The clays may be layered double hydroxides or natural or converted clays such as those known to a person skilled in the art as decolourizing earths. Preferably, at least one adsorbent is an activated carbon. Therefore, when the adsorption section comprises a single type of adsorbent, said adsorbent is an activated carbon and, when the adsorption section comprises two or more different adsorbents, one adsorbent is an activated carbon and the other(s) is/are another activated carbon, an alumina and/or a clay, preferably an activated carbon and/or a clay, more particularly a clay.

Preferably, each adsorbent has a pore volume (Vp), determined by mercury porosimetry, of greater than or equal to 0.25 ml/g, preferentially greater than or equal to 0.40 ml/g, with preference greater than or equal to 0.50 ml/g, and preferably less than or equal to 5 ml/g.

Preferably, each adsorption section of substep e1) is implemented:
in flow-through fixed-bed mode, that is to say in at least one adsorber comprising a fixed bed of adsorbent(s), in particular at least one column of adsorbent(s), with operation being possible in ascending or descending mode, preferably in ascending mode, or
in stirred mode, advantageously in at least one continuous stirred reactor, also known as continuous stirred tank reactor (CSTR).

In the case where the adsorption section is implemented in stirred mode in at least one CSTR-type stirred reactor, the reactor(s) is/are followed by a filtration system for recovering said adsorbent(s) which is/are in suspension in the liquid treated. Preferably, the adsorption section is implemented in flow-through fixed-bed mode.

Preferably, in the case where each adsorption section comprises at least two different adsorbents, the adsorbents may be:
all present in each column of the adsorption section(s), in a mixture or in successive fixed beds, or
each used in an adsorption subsection of the adsorption section(s), the subsections being placed in series with respect to one another, each adsorption subsection comprising, preferably consisting of, between one and four, preferably between two and four, fixed-bed adsorbent columns.

Very advantageously, each adsorption section or each of the subsections comprises two or more fixed-bed columns, in particular at least two fixed-bed columns, preferably between two and four fixed-bed columns, of the same adsorbent(s). When the adsorption section or the adsorption subsection comprises two columns of the same adsorbent(s), the adsorption section may operate according to a “swing” operating mode in which one of the columns is on-line while the other column is in reserve. When the adsorbent in the on-line column is spent, this column is isolated, while the column in reserve is placed on-line. The spent adsorbent of the isolated column may then be regenerated in situ and/or replaced with fresh adsorbent, to be placed back on-line again once the other column has been isolated. Another mode for operating the adsorbent columns is to have at least two columns operating in series: when the adsorbent of the lead column (that is, the first column in the series) is spent, this first column is isolated and the spent adsorbent is regenerated in situ or replaced with fresh adsorbent, said column then being placed back on-line in the last position of the series of columns and so on.

This operation is referred to as “lead-lag”. Very preferably, each adsorption section implements at least two columns of the same adsorbent, preferably two to four columns of the same adsorbent, preferentially in two columns of the same adsorbent, operating in “lead-lag” mode.

The combination of at least two columns of the same adsorbent makes it possible in particular to remedy the, possibly rapid, saturation and/or clogging of the adsorbent. Specifically, the presence of at least two columns of adsorbent facilitates the replacement and/or the regeneration of the adsorbent, advantageously without halting the adsorption unit (e1), or even the process, thus making it possible to reduce the risks of clogging and prevent shutdowns of the unit due to saturation of the adsorbent, to manage costs and to limit the consumption of adsorbent, while ensuring continuous production of purified diester monomers. This combination of at least two columns of adsorbent, in particular operating in “lead-lag” mode, also makes it possible to maximize the adsorption capacity of said adsorbent.

In a very particular embodiment of the invention, in which each adsorption section comprises two different adsorbents, each adsorption section very preferentially comprises a first subsection comprising at least two, preferably between 2 and 4, fixed-bed columns of activated carbon, operating in swing or in lead-lag mode, and a second subsection comprising at least two, preferably between 2 and 4, fixed-bed columns of another adsorbent, preferably chosen from another activated carbon or a clay, operating in particular in swing or in lead-lag mode, and placed upstream or downstream of the first subsection of fixed-bed activated carbon columns.

Preferably, each adsorbent is in the form of granules, extrudates or powder. Preferably, each adsorbent is:
in the form of granules or of extrudates when the adsorption section(s) is/are implemented in through-flow fixed-bed mode, and
in the form of powder when the adsorption section(s) is/are implemented in CSTR-type stirred reactor.

The size of said at least one adsorbent, in particular when it is in the form of granules or extrudates, is such that the smallest dimension of said at least one adsorbent (corresponding to the diameter of the circle circumscribed on the basis of the granules or polylobal extrudates or to the diameter of the cylinder circumscribed on the cylindrical basis of the extrudates of cylindrical type; this dimension also being called “diameter”) is preferably between 0.1 and 5 mm, preferentially between 0.3 and 2 mm. For example, the activated carbon extrudates of 0.8 mm diameter sold by Cabot Norit or the granules within the size range between 0.4 and 1.7 mm sold by Chemviron may be suitable as adsorbent in the adsorption section of substep e1).

The adsorption substep e1) may advantageously also comprise a phase of regeneration of said adsorbent(s).

An adsorption-pretreated monomer effluent is obtained at the end of each adsorption section and advantageously feeds the crystallization substep e2). When substep e1) implements two or more adsorption sections, i.e. between two and ten, preferably between two and four adsorption sections, the monomer effluents generated at the outlet of the adsorption sections may advantageously be recombined to constitute an adsorption-pretreated monomer effluent which then feeds crystallization substep e2).

Crystallization Substep e2) Advantageously, crystallization substep e2) implements at least one solids production section and at least one solid-liquid separation section. Crystallization substep e2) makes it possible to obtain a decolourized purified diester monomer effluent and a spent solvent effluent.

Advantageously, crystallization substep e2) implements one or more crystallization or precipitation operations and one or more solid-liquid separation operations. According to a particular embodiment of the invention, crystallization substep e2) implements a solids production section as described hereinafter, followed by a solid-liquid separation section as detailed further below. According to another particular embodiment of the invention, crystallization substep e2) implements two or more solids production sections, preferably between two and five solids production sections, as described hereinafter, each of the solids production sections being followed by a solid-liquid separation section as detailed further below.

The solids production section of substep e2) is fed with the prepurified monomers effluent obtained from step d) or with the adsorption-pretreated monomer effluent obtained from substep e1), preferably with the adsorption-pretreated monomer effluent obtained from substep e1). Optionally, the solids production section may also be fed with a crystallization solvent which is identical to or different from the solvent introduced into the mixing section of substep e1). The crystallization solvent is advantageously chosen from water, monoalcohols, diols, ethers, aldehydes, esters, hydrocarbons and mixtures of at least two of these compounds belonging to the same chemical family or different chemical families. Preferably, said crystallization solvent is chosen from water, monoalcohols having between 1 and 12 carbon atoms, such as methanol or ethanol, diols having between 1 and 12 carbon atoms, aromatic hydrocarbons, for example monoaromatic compounds or mixtures of monoaromatic compounds, and mixtures of at least two of said compounds. Very advantageously, said crystallization solvent is water, a monoalcohol having between 1 and 12 carbon atoms, such as methanol or ethanol, a diol having between 1 and 12 carbon atoms, such as ethylene glycol, a monoaromatic compound, for example xylene, or a mixture thereof. Preferably, the crystallization solvent is water, a diol having between 1 and 12 carbon atoms, preferably ethylene glycol, or mixtures thereof.

According to a preferred embodiment of the invention, the crystallization solvent comprises, preferably consists of, all or part of a solvent effluent obtained from the spent solvent effluent obtained at the end of the solid-liquid separation section of substep e2), which may or may not be purified, optionally supplemented by a supply of solvent external to the process according to the invention.

Preferably, when crystallization solvent is introduced in substep e2), the amount of crystallization solvent introduced into the solids production section is adjusted such that the prepurified monomers effluent which feeds substep e1) represents between 1% and 75% by weight, preferentially between 5% and 45% by weight, preferably between 15% and 35% by weight, of the total weight of the mixture in said solids production section (i.e. the mixture comprising the prepurified monomers effluent, the solvent introduced in substep e1) and the crystallization solvent introduced in substep e2)).

Prior to being introduced into the solids production section, all or part of the crystallization solvent may be heated, preferably to the temperature at which the adsorption section is operated, or cooled and in particular brought to a temperature preferably of between 0 and 120° C., preferably between 5 and 100° C., and with preference between 10 and 90° C.

Advantageously, the solids production section of substep e2) is operated at a temperature of (i.e. such that the temperature of the effluent obtained from said solids production section is) between 0 and 100° C., preferably between 5 and 80° C., and with preference between 10 and 70° C. More precisely, in the solids production section, the adsorption-pretreated monomer effluent, optionally in a mixture with the crystallization solvent, is cooled from the temperature at which the adsorption section is operated, i.e. from a temperature of between 50 and 200° C., preferably between 70° C. and 170° C., preferentially between 80 and 150° C., with preference between 80 and 120° C., to a temperature of between 0 and 100° C., preferably between 5 and 80° C., and with preference between 10 and 70° C.

The cooling may be implemented according to any method known to a person skilled in the art. For example, in particular in batch mode, the cooling of the temperature may be realized without regulation of the lowering of the temperature (i.e. without an imposed temperature ramp; thus only the initial and final temperatures are controlled) or according to at least one decreasing temperature ramp, in particular according to a decreasing temperature ramp of between 5 and 30° C./hour and more particularly between 8 and 15° C./hour, or else according to both modes chained together in succession, i.e. without control for one part of the cooling and according to a decreasing temperature ramp for another part of the cooling. According to another example, the cooling may simply be due to the introduction of the stream to be cooled, i.e. the adsorption-pretreated monomer effluent obtained from adsorption step e1) or the mixture comprising the adsorption-pretreated monomer effluent and the crystallization solvent, into a reservoir having a volume which is advantageously adapted to the flow rate of the stream to be cooled and which is held at a temperature of between 0 and 100° C., preferably between 5 and 80° C., and with preference between 10 and 70° C.

The solids production section is advantageously operated at a pressure of between 0.00001 and 1.00 MPa, preferably between 0.0001 and 0.50 MPa, and with preference between 0.001 and 0.20 MPa. According to a particular embodiment of the invention, the solids production section is operated under vacuum, preferably at a pressure of between 0.0001 and 0.10 MPa, preferentially between 0.001 and 0.01 MPa. According to another particular embodiment, the solids production section is advantageously operated in a jacketed reactor, at a pressure of between 0.01 and 1.00 MPa, preferably between 0.05 and 0.20 MPa, with preference at atmospheric pressure, i.e. at 0.10 MPa.

Advantageously, the solids production section has the purpose of rendering solid, i.e. crystallizing or precipitating, at least in part, the diester monomer, preferably the BHET, in particular present in the adsorption-pretreated monomer effluent obtained from substep e1).

Thus, the solids production section comprises, and preferably consists of, a precipitation or crystallization phase implemented by any precipitation or crystallization techniques known to a person skilled in the art. The solids production section is preferably a section for crystallization, for example by cooling or by concentration, which is implemented in any equipment known to a person skilled in the art, as defined, for example, in the Journal Techniques de L'ingénieur, “Cristallisation industrielle—Aspects pratiques” [Industrial Crystallization—Practical Aspects], ref. J2788 V1, followed by a liquid-solid separation.

According to a preferred embodiment of the invention, water, as crystallization solvent, is mixed with the adsorption-pretreated monomer effluent obtained from substep e1) and the solids production section is operated under conditions such that the temperature of the effluent obtained from said solids production section is between 5 and 50° C., preferably between 10 and 40° C.

According to another preferred embodiment of the invention, the crystallization solvent introduced and mixed with the adsorption-pretreated monomer effluent obtained from substep e1) is ethylene glycol and the solids production section is operated under conditions such that the temperature of the effluent obtained from said solids production section is between 5 and 50° C., preferably between 10 and 40° C.

Advantageously, said section for solids production, preferably by crystallization, comprises one or more crystallization operations, operating in series or in parallel, which is/are carried out batchwise or continuously, preferably continuously.

The solids production section makes it possible to obtain a heterogeneous effluent comprising a diester monomer solid phase and a liquid phase. The heterogeneous effluent is advantageously sent to the solid-liquid separation section.

In the solid-liquid separation section, the diester monomer, preferably the BHET, advantageously in solid form, and in particular in the form of crystals, is separated from the liquid phase comprising all or part of the solvent introduced into the mixing section of substep e1) and the crystallization solvent optionally introduced into the solids production section. The solid-liquid separation section advantageously implements any means of solid-liquid separation known to a person skilled in the art, in particular at least one filtration, decantation and/or centrifugation system. The solid diester monomer thus separated constitutes the decolourized purified diester monomer effluent, the liquid phase constituting the spent solvent effluent.

According to a particular embodiment of the invention, the decolourized purified diester monomer effluent, recovered in solid form preferably by filtration or centrifugation, may additionally advantageously undergo all or some of the following operations, carried out one or more times without a predefined chronological order: rinsing with a solvent, identical to or different from the solvent feeding the mixing section or optionally the solids production section; additional filtration or centrifugation; removal of the residual solvent by any method known to a person skilled in the art, for example by evaporative drying; shaping, for example into powder or granules; and storage of the solid.

According to another embodiment of the invention, the decolourized purified diester monomer effluent is recovered, preferably by filtration or centrifugation, in the solid-liquid separation section and then is directly sent (i.e. without a phase of storage of the solid) to a polymerization step known to a person skilled in the art, optionally with, prior to the polymerization reaction, an operation of rinsing with water or a diol effluent, for example an ethylene glycol effluent, preferably an operation of rinsing with water, of the solid purified diester monomer effluent, followed by heating of the rinsed solid for the purposes of melting.

According to another particular embodiment of the invention, purification step e) may comprise a crystallization substep e2) followed by an adsorption substep e1), as described above, in particular in which:
substep e2) is fed with the prepurified monomers effluent obtained from step d) and optionally the crystallization solvent;
substep e2) produces a solid which feeds substep e1);
the solid produced on conclusion of substep e2) is dissolved in the solvent of the mixing section of substep e1), to obtain a solution;
the solution obtained at the outlet of the mixing section of substep e1) feeds the adsorption section(s) of substep e1);
a decolourized purified diester monomer effluent in liquid form is obtained, the decolourized purified diester monomer then being able to be precipitated and/or recrystallized according to any one of the methods known to a person skilled in the art, so as to obtain a decolourized purified diester monomer effluent in solid form.

Advantageously, the decolourized purified diester monomer effluent, obtained on conclusion of the process according to the invention, preferably comprises at least 90% by weight, preferentially at least 95% by weight, with preference at least 98% by weight, of diester monomer (i.e. of the product targeted by the process according to the invention), preferably BHET. The decolourized purified diester monomer effluent, obtained on conclusion of the process according to the invention, very advantageously comprises less than 5% by weight, preferably less than 1% by weight and preferentially less than 0.5% by weight, of impurities of the type of esters of dicarboxylic acid with at least one diol dimer or trimer, such as ester compounds derived from diethylene glycol (for example 2-(2-hydroxyethoxy)ethyl 2-hydroxyethyl terephthalate).

Very advantageously, the decolourized purified diester monomer effluent, obtained on conclusion of the process according to the invention, is a white solid. Purification step e), which comprises a phase of treatment by adsorption of a monomer solution and then a phase of crystallization of said monomer, thus makes it possible to satisfactorily decolourize the prepurified diester monomers effluent obtained from step d). Specifically, the dyes possibly present in the prepurified monomers effluent obtained from step d) remain either trapped by the adsorbent in the adsorption section or dissolved in the solvent, or the mixture of solvents (solvent introduced in substep e1) and that optionally introduced in substep e2)), during the solids production operation and are thus concentrated in the spent solvent effluent.

The decolourized purified diester monomer effluent, obtained on conclusion of purification step e) of the process of the invention, is thus advantageously a white solid, as viewed with the eye.

The decolourized purified diester monomer effluent, obtained on conclusion of step e), may be characterized by UV-visible spectrometry in order to identify the presence of absorption bands in the visible range, in particular between 400 and 800 nm. According to this characterization method, the decolourized purified diester monomer effluent is preferably characterized by UV-visible spectrometry, in particular between 400 and 800 nm, advantageously in liquid medium, that is to say after dissolution in a suitable solvent, preferably at between 0.1% and 10% by mass, at ambient temperature (typically between 15 and 30° C., in particular between 20 and 25° C.), using a conventional laboratory counter-top UV-visible spectrometer. Ethanol may be used as a suitable solvent, allowing dissolution of a sample of the decolourized purified diester monomer effluent. A conventional cuvette with 1 cm or 1 inch optical path length may be used.

Preferably, the UV-visible spectrum of the decolourized purified diester monomer effluent is determined with the aid of a solution of the decolourized purified diester monomer effluent prepared at 5% by mass in ethanol and of a cuvette with a 1 inch optical path length. According to this method, the decolourized purified diester monomer effluent obtained by the process according to the invention advantageously exhibits a spectrum not displaying any significant absorption band (that is to say distinguishable from background noise) within the range of visible wavelengths, that is to say between 400 and 800 nm.

The decolourized purified diester monomer effluent obtained on conclusion of step e) may also be characterized according to a colourimetry method such as described in ASTM D6290 2019.

The illuminant chosen is illuminant D65, measurements are carried out in reflection and in specular excluded mode, 10⁰ standard observer. The measurements are expressed in the CIE L*a*b* reference system. According to the colourimetry method, the decolourized purified diester monomer effluent obtained by the process according to the invention advantageously exhibits a CIE L*a*b* reference system with:
a lightness (or luminance) parameter L* of close to 100, more particularly greater than 90.00 and preferably greater than 92.00 (100.00 being the maximum);
a parameter a* (corresponding to a green-red axis) of close to 0, more particularly between −1.50 and +1.50 and preferably between −1.00 and +1.00; and
a parameter b* (corresponding to a blue-yellow axis) of close to 0, more particularly between −2.50 and +2.50, more particularly between −1.00 and +1.50.

The spent solvent effluent comprises all or part of the solvent introduced into the mixing section of substep e1) and of the crystallization solvent optionally introduced into the solids production section. It also advantageously comprises dyes and/or other residual impurities. Preferably, the spent solvent effluent comprises less than 20% by weight, preferentially less than 15% by weight, with preference less than 10% by weight and with preference less than 5% by weight, of diester monomer (i.e. of product targeted by the process according to the invention), preferably of BHET monomer.

The spent solvent effluent may then be recycled to the mixing section of substep e1) or one of steps a) and/or b) of the process when the purified solvent is a diol of the same nature as that used for the depolymerization reaction, and/or optionally to section e2) as crystallization solvent. The spent solvent effluent may also be treated, at least in part, so as in particular to separate the dyes and/or impurities, for example by adsorption, and thus to recover a purified solvent which may then be recycled to the mixing section of substep e1) or one of steps a) and/or b) of the process when the purified solvent is a diol of the same nature as that used for the depolymerization reaction, and/or optionally to substep e2) as crystallization solvent. The spent solvent effluent may also undergo, in addition to the separation of the dyes and/or impurities, an operation for separation of solvents, for example by distillation or decantation, when a crystallization solvent is introduced in substep e2) and the crystallization solvent is different from the solvent introduced in the mixing section of substep e1), to then obtain two separate solvents, one able to be recycled to the mixing section of substep e1) and the second able to be recycled to the solids production section of substep e2).

The decolourized purified diester monomer effluent obtained on conclusion of the process according to the invention may thus feed, directly or indirectly, a polymerization step known to a person skilled in the art for the purpose of producing a polyester polymer, preferably PET or a PET-based copolyester, which is indistinguishable from the corresponding virgin resin. Said polymerization step may also be fed, in addition to the decolourized purified diester monomer effluent, with ethylene glycol, terephthalic acid or dimethyl terephthalate, or any other monomer, in accordance with the targeted (co)polymer.

The following figures and examples illustrate the invention without limiting the scope thereof.

### EXAMPLES

In the examples below, the steps a) of conditioning, b) of depolymerization, c) of separation of the diol and d) of separation of the monomer are identical and are described below. The only variation is in the purification step e) between the processes of Example 1 (in accordance with the invention) and Example 2 (not in accordance).

A polyester feedstock comprising, in particular, 20% by weight of opaque PET is obtained from collection and sorting channels for treatment. 4 kg/h of flakes of said polyester feedstock comprising 20% by weight of opaque PET, itself containing 6.2% by weight of TiO₂ pigment, are brought to a temperature of 250° C. and then injected with 11.5 kg/h of ethylene glycol (MEG) into a first stirred reactor which is maintained at 250° C., and then into a second and a third stirred reactor, which are maintained at 220° C. The reactors are maintained at a pressure of 0.4 MPa. The residence time, defined as the ratio of the liquid volume in the reactor to the sum of the liquid volume flow rates entering the reactor, is set at 20 min in the first reactor and 2.1 h in the second and third reactors. At the outlet of the third reactor, the reaction effluent consists of 67.7% by weight of diol composed very predominantly of ethylene glycol (MEG) (i.e. comprising 95% by weight or more of MEG), 25.8% by weight of diester monomer composed very predominantly of bis(2-hydroxyethyl) terephthalate (BHET) (i.e. comprising 95% by weight or more of BHET), 0.32% by weight of TiO₂, and 6.1% by weight of heavy compounds including inter alia dimers and/or oligomers of BHET.

The diol present in the reaction effluent is separated by evaporation in a succession of two flash vessels at temperatures ranging from 180° C. to 120° C. and pressures from 0.04 MPa to 0.004 MPa, followed by a thin-film evaporator operated at 175° C. and 0.0005 MPa. At the end of this evaporation step, an MEG-rich stream of 10.46 kg/h and a BHET-rich liquid stream of 5.02 kg/h are recovered. The MEG-rich stream, corresponding to a diol effluent, is sent to a step of purification by distillation to produce a purified MEG stream which may, at least on the one hand, be recycled to the depolymerization reactor. The BHET-rich liquid stream, corresponding to the liquid monomers effluent, consists of 79.6% by weight of BHET diester monomer, 0.6% by weight of MEG and 1.0% by weight of TiO₂ and 18.8% by weight of heavy compounds including dimers of BHET.

The BHET-rich liquid stream is then injected into a short-path evaporator, also referred to as short-path distillation, which is operated at a pressure of 20 Pa. A hot oil at 215° C. enables the evaporation of the BHET, which is subsequently condensed in the short-path evaporator at 130° C. to give a liquid stream of prepurified BHET (corresponding to the prepurified monomers effluent). The residence time in the short-path evaporator is 1 min. The liquid stream of prepurified BHET represents a flow rate of 3.8 kg/h and is recovered as distillate from the short-path evaporator. It consists of 99% by weight of BHET diester monomer and is devoid of any trace of TiO₂. A heavy residue with a flow rate of 1.19 kg/h is recovered as a residue from the short-path evaporator and consists of 16.7% by weight of BHET diester monomer, 79.2% by weight of BHET oligomers and 4.1% by weight of TiO₂. A portion of the heavy residue may be purged, while the other portion may be recycled to the reaction step.

### Example 1—in Accordance

### Adsorption Substep e1)

The liquid stream of prepurified BHET containing 99% by weight of BHET diester is compressed to 0.15 MPa and feeds, at a flow rate by weight of 3.8 kg/h, a mixing section which is also fed with a stream of water. The water feed flow rate is adjusted such that said liquid stream of prepurified BHET represents 50% by weight of the mixture (liquid stream of prepurified BHET+water). Said mixing section is operated at 90° C., at a pressure of 0.15 MPa.

The resulting mixture then feeds an adsorption section consisting of two columns each filled with an adsorbent (i.e. with a fixed bed of adsorbent). The adsorption section is operated at 90° C., at a pressure of 0.15 MPa. One column is placed on stream (i.e. it is in operation), the other remaining in reserve. The adsorbent used to fill the two columns is an activated carbon consisting of cylindrical extrudates with a diameter of 0.8 mm, reference ROY 0.8 from Cabot Norit.

The residence time is fixed at 40 minutes, in one column. The empty column linear velocity is 2.4 cm/min.

### Crystallization Substep e2)

A batch of 780 g of the liquid stream obtained on conclusion of adsorption substep e1) is mixed in a stirred tank with water such that the amount by weight of the liquid stream of prepurified BHET introduced in substep e1) represents 20% by weight of the final mixture and the amount of water introduced in substep e1) and substep e2) represents 80% by weight of the final mixture, until a temperature of 60° C. is reached. The mixture, kept under stirring, is cooled to 50° C. over 1 h and is then progressively cooled according to a ramp of 12° C./hour down to 20° C.

Solid particles form over the course of the cooling to afford a suspension of solids in a liquid comprising predominantly water. The suspension obtained at 20° C. is then filtered to recover a solid cake and a coloured liquid filtrate. The solid cake is rinsed with 1.5 l of water. The rinsed solid cake is recovered and then dried overnight under vacuum at 40° C. to afford 320 g of a white solid containing 99% by weight of BHET diester (determination of the composition by liquid chromatography).

The recovered solid is white. Measurement by UV-visible spectrometry is performed on a BHET solution prepared with a sample of the obtained white solid dissolved at 5% by weight in ethanol. The measurement by UV-visible spectrometry is performed using a Hach DR3900 laboratory counter-top UV-visible spectrometer in a cuvette with an optical path length of one inch. The UV-visible spectrum obtained does not display any significant absorption band in the wavelength range between 400 and 800 nm (cf. FIG. 3).

Colourimetry measurements are also performed on the solid BHET obtained, in accordance with the method ASTM D6290 2019. A sample of 5 g of solid BHET product is reduced to powder by grinding in a mortar. The 5 g of ground BHET are placed in a cuvette made of optical quality glass, with a diameter of 34 mm. The measurements are performed in reflection using a Konica Minolta CM-2300d colourimeter and SpectraMagic NX software, under the following conditions: illuminant D65, specular excluded, 100 standard observer. The measurements are expressed in the CIE L*a*b* reference system. The result is obtained by averaging the values obtained for 10 measurements performed on the sample. The results are presented in Table 1.

### Example 2—not in Accordance

A batch of 780 g of the liquid stream of prepurified BHET obtained at the outlet of the short-path distillation is mixed in a stirred tank with water, so as to achieve a final content of 20% by weight of the prepurified BHET liquid stream and 80% by weight of water, and a final temperature of 60° C. The mixture, kept under stirring, is cooled to 50° C. over 1 h and is then progressively cooled according to a ramp of 12° C./min down to 20° C.

Solid particles form over the course of the cooling to afford a suspension of solids in a liquid composed very predominantly of water. The suspension is then filtered to recover a solid cake and a coloured liquid filtrate. The solid cake is rinsed with 1.5 l of water. The rinsed solid cake is recovered and then dried overnight under vacuum at 40° C. to afford 320 g of a white solid containing 99% by weight of BHET diester.

Measurement by UV-visible spectrometry is performed on a BHET solution prepared with a sample of the obtained white BHET solid dissolved at 5% by weight in ethanol. The measurement by UV-visible spectrometry is performed using a Hach DR3900 laboratory counter-top UV-visible spectrometer in a cuvette with an optical path length of one inch. The UV-visible spectrum obtained displays significant absorption bands in the wavelength range between 400 and 800 nm (cf. FIG. 3).

Colourimetry measurements are also performed on the solid BHET obtained, in accordance with the method ASTM D6290 2019. A sample of 5 g of solid BHET product is reduced to powder after grinding in a mortar. The 5 g of BHET are placed in a cuvette made of optical quality glass, with a diameter of 34 mm. The measurements are performed in reflection using a Konica Minolta CM-2300d colourimeter and SpectraMagic NX software, under the following conditions: illuminant D65, specular excluded, 10⁰ standard observer. The measurements are expressed in the CIE L*a*b* reference system. The result is obtained by averaging the values obtained for 10 measurements performed on the sample. The results are presented in Table 1.

TABLE 1
Results of the colourimetry measurements according to the CIE
L*a*b* reference system, obtained for the solids produced
on conclusion of the processes according to Examples 1 and 2.

| | L* | a* | b* |
|---|---|---|---|
| Example 1 (in accordance) | 93.02 | 0.03 | 1.00 |
| Example 2 (not in | 90.02 | 1.21 | 2.22 |
| accordance) | | | |

The results of the measurements (by UV-visible spectrometry and by colourimetry) performed on the BHETs obtained from the processes described in Examples 1 and 2 show:
the absence of absorption band in the visible range (400-800 nm) for the BHET obtained from the process of Example 1, whereas the BHET obtained from the process of Example 2 displays significant absorption bands in the wavelength range between 400 and 800 nm (cf. FIG. 3);
L*a*b* colourimetry values reflecting the obtaining of a product according to the process described in Example 1 which is “whiter” than that obtained by the process described in Example 2, or at the least L*a*b* colourimetry values which are in accordance with the targeted L*a*b* values, since:
the solid BHET obtained according to the process of Example 1 has an L* value of 93.02, which is greater than 92.00 and greater than that of the solid BHET obtained according to the process of Example 2 (L*=90.02),
the solid BHET obtained according to the process of Example 1 (in accordance) has a* and b* values respectively of 0.03 and 1.00, which are closer to 0 in absolute value than those obtained for the solid BHET obtained from the process of Example 2 (a*=1.21 and b*=2.22). Thus, the process described in Example 1, in accordance with the invention, which comprises a step of purification of the BHET with an adsorption step followed by a crystallization step, makes it possible to obtain a decolourized purified BHET of better quality (since it is better decolourized) than a BHET obtained on conclusion of a process as described in Example 2 (not in accordance) comprising only a crystallization step as purification step.

1. A process for depolymerization of a polyester feedstock comprising polyethylene terephthalate, the process comprising:
a) a conditioning step fed at least with said polyester feedstock, to produce a conditioned feedstock stream;
b) a step of depolymerization by glycolysis, which is fed at least with the conditioned feedstock stream, and is operated at a temperature of between 150 and 300° C., with a residence time of between 0.1 and 10 h, in the presence of diol with a ratio by weight between the total amount of diol present in step b) and the amount of diester contained in the conditioned feedstock stream of between 0.3 and 8.0, to produce a reaction effluent;
c) a step of separation of the diol, which is fed at least with the reaction effluent obtained from step b), and is operated at a temperature of between 60 and 250° C. and at a pressure lower than that of step b), to produce at least a diol effluent and a liquid monomers effluent,
said step of separation of the diol being implemented in a gas-liquid separation section or a succession of from two to five successive gas-liquid separation sections, each of the gas-liquid separation sections producing a gas effluent and a liquid effluent, the liquid effluent from the preceding section feeding the subsequent section, the liquid effluent obtained from the final gas-liquid separation section constituting the liquid monomers effluent, the gas effluent(s) being recovered so as to constitute said diol effluent(s);
d) a step of separation of the liquid monomers effluent obtained from step c) into a heavy impurities effluent and a prepurified monomers effluent, which is operated at a temperature of less than 250° C. and a pressure of less than 0.001 MPa, with a liquid residence time of less than 10 min; and
e) a step of purification of the prepurified monomers effluent, comprising an adsorption substep e1) and a crystallization substep e2), and producing at least one decolorized purified diester monomer effluent, wherein
adsorption substep e1) is operated at a temperature of between 50 and 200° C. and a pressure of between 0.1 and 1.0 MPa and implements at least one section for mixing with a solvent and at least one section for adsorption in the presence of at least one adsorbent,
crystallization substep e2) implements a solids production section, operated at a temperature of between 0 and 100° C. and at a pressure of between 0.00001 and 1.00 MPa, followed by a solid-liquid separation section.

2. The process according to claim 1, wherein step e) of purification of the prepurified monomers effluent comprises an adsorption substep e1) followed by a crystallization substep e2), producing at least one decolorized purified diester monomer effluent and a spent solvent effluent, wherein
adsorption substep e1) is operated at a temperature of between 50 and 200° C. and a pressure of between 0.1 and 1.0 MPa and implements at least one section for mixing the prepurified monomers effluent obtained from step d) with a solvent and at least one section for adsorption in the presence of at least one adsorbent, to obtain an adsorption-pretreated monomer effluent,
crystallization substep e2) implements a solids production section, fed at least with the adsorption-pretreated monomer effluent and operated at a temperature of between 0 and 100° C. and at a pressure of between 0.00001 and 1.00 MPa, followed by a solid-liquid separation section, to produce the decolorized purified diester monomer effluent and the spent solvent effluent.

3. The process according to claim 1, wherein the amount of solvent introduced into the mixing section of substep e1) is adjusted such that the prepurified monomers effluent obtained from step d) or a solid produced on conclusion of substep e2) represents between 20% and 90% by weight, preferentially between 30% and 80% by weight, preferably between 50% and 75% by weight and more preferably still between 40% and 60% by weight, of the total weight of the mixture of said mixing section.

4. The process according claim 1, wherein the solvent which feeds the mixing section of substep e1) is chosen from water, alcohols, diols, for example ethylene glycol and mixtures thereof, and preferably diols, for example ethylene glycol, water and mixtures thereof.

5. The process according to claim 1, wherein substep e1) is operated at a temperature of between 70 and 170° C., preferably between 80 and 150° C.

6. The process according to claim 1, wherein substep e1) is operated at a pressure of between 0.1 and 0.8 MPa, preferably between 0.1 and 0.5 MPa.

7. The process according to claim 1, wherein at least one adsorbent of the adsorption section of substep e1) is an activated carbon.

8. The process according to claim 1, wherein the solids production section is operated at a temperature of between 5 and 80° C., preferably between 10 and 70° C.

9. The process according to claim 1, wherein the solids production section is operated at a pressure of between 0.0001 and 0.50 MPa, preferably between 0.001 and 0.20 MPa.

10. The process according to claim 1, wherein the solids production section is fed with a crystallization solvent which is identical to or different from the solvent introduced into the mixing section of substep e1), and wherein the amount of crystallization solvent introduced into the solids production section is adjusted such that the prepurified monomers effluent which feeds substep e1) represents between 1% and 75% by weight, preferentially between 5% and 45% by weight, preferably between 15% and 35% by weight, of the total weight of the mixture in said solids production section.

11. The process according to claim 1, wherein the polyester feedstock comprises at least 50% by weight, preferably at least 70% by weight, with preference at least 90% by weight, of polyethylene terephthalate, and in particular comprises opaque PET, colored PET, multilayer PET, or mixtures thereof.

12. The process according to claim 1, wherein the polyester feedstock comprises between 0.1% and 10% by weight of pigments, preferably between 0.1% and 5% by weight of pigments, and preferably between 0.005% and 1% of dyes, in particular between 0.01% and 0.2% by weight of dyes.

13. The process according to claim 1, wherein step a) is implemented at a temperature of between 200 and 300° C., preferably between 250 and 290° C., and preferably at a pressure of between 0.1 MPa and 20 MPa, with preference between 0.15 MPa and 10 MPa.

14. The process according to claim 1, wherein step a) is fed with a diol stream with a ratio by weight of the diol stream in relation to the polyester feedstock of between 0.03 and 6.00, preferably between 0.05 and 5.00, preferentially between 0.10 and 4.00, with preference between 0.50 and 3.00.

15. The process according to claim 1, wherein the ratio by weight between the total amount of diol present in step b) and the amount of diester contained in the conditioned feedstock stream is between 1.0 and 7.0, preferably between 1.5 and 6.0.

16. The process according to claim 1, wherein the amount of solvent introduced into the mixing section of substep e1) is adjusted such that the prepurified monomers effluent obtained from step d) or a solid produced on conclusion of substep e2) represents between 30% and 80% by weight of the total weight of the mixture of said mixing section.

17. The process according claim 1, wherein the solvent which feeds the mixing section of substep e1) is selected from ethylene glycol, water, and mixtures thereof.

18. The process according to claim 1, wherein substep e1) is operated at a temperature of between 80 and 150° C.

19. The process according to claim 1, wherein substep e1) is operated at a pressure of between 0 0.1 and 0.5 MPa.

20. The process according to claim 1, wherein the solids production section is operated at a temperature of between 10 and 70° C.
